(19) United States
(12) Patent Application Publication
(10) Pub. No.: US 2022/0071505 A1
(43) Pub. Date: Mar. 10, 2022

(54) INTERCHANGEABLE SENSOR AND METHOD

(71) Applicant: Know Labs, Inc., Seattle, WA (US)

(72) Inventor: Phillip BOSUA, Seattle, WA (US)

(21) Appl. No.: 17/465,112

(22) Filed: Sep. 2, 2021

Related U.S. Application Data
(60) Provisional application No. 63/075,930, filed on Sep. 9, 2020.

(51) Int. Cl. A61B 5/0507; A61B 5/00; A61B 5/145

(52) U.S. Cl. CPC A61B 5/0507; A61B 5/6824; A61B 5/0075; A61B 5/14532; A61B 5/1451; A61B 5/14546

(57) ABSTRACT

An interchangeable sensor that can perform detection via spectroscopic techniques using non-optical frequencies such as in the radio or microwave frequency bands of the electromagnetic spectrum. The interchangeable sensor is removably disposable on a user wearable sensing assembly that can be worn by a user for non-invasively detecting an analyte in the user. The sensor can also be removably installed on a non-user wearable sensing assembly for performing a detection function using the sensor installed on the non-user wearable sensing assembly.

### FIELD

This technical disclosure relates to apparatus, systems and methods of performing detection via spectroscopic techniques using non-optical frequencies such as in the radio or microwave frequency bands of the electromagnetic spectrum. More specifically, this disclosure relates to an interchangeable sensor that can be used in a user wearable sensor assembly, then removed from the user wearable sensor assembly and used as a sensor in a non-user wearable sensor assembly to perform a different detection.

### BACKGROUND

There is interest in being able to detect and/or measure an analyte within a target. One example is measuring glucose in biological tissue. One non-limiting example is measuring glucose in biological tissue. U.S. Pat. No. 10,548,503 discloses an example of the use of a sensor that uses radio or microwave frequency bands of the electromagnetic spectrum in in vivo medical diagnostics. U.S. Pat. No. 10,548,503 is incorporated herein by reference in its entirety.

### SUMMARY

This disclosure relates generally to apparatus, systems and methods of implementing an interchangeable, form factor agnostic sensor that operates via spectroscopic techniques using non-optical frequencies such as in the radio or microwave frequency bands of the electromagnetic spectrum. The sensor is removably disposable on a user wearable sensing assembly that can be worn by a user for non-invasively detecting an analyte in the user. The sensor can be removed from the user wearable sensing assembly and removably installed on a non-user wearable sensing assembly to perform another detection function. For example, the detection function performed by the non-user wearable sensing assembly can include, but is not limited to, detecting an analyte in a sample material or detecting a characteristic of a substrate or other material. In another embodiment, the entire user wearable sensing assembly can be removably installed on the non-user wearable sensing assembly.

The user wearable sensing assembly can be configured to be worn at any location on the user. In one non-limiting example, the user wearable sensing assembly can be configured to be worn on the user's arm, for example the user's wrist, with a wrist strap that is directly or indirectly detachably fastenable to the sensor. In some embodiments, the user wearable sensing assembly can be worn by an animal to detect an analyte within the animal.

The non-user wearable sensing assembly can be any sensing assembly that is not user wearable and that is used to perform a detection function. For example, the detection function can include detecting an analyte or characteristic in a sample or substrate that is separate from the user. The analyte in the sample can be a different analyte than the analyte detected by the user wearable sensing assembly. In another embodiment, the analyte in the sample can be the same analyte that is detected by the user wearable sensing assembly. The sample can be a liquid, a gas, a solid, a semi-fluid, a semi-solid, a gel, and combinations thereof, human or non-human, animal or non-animal, biological or non-biological, that contains the analyte(s) that one may wish to detect. Examples of samples include, but are not limited to, human tissue, animal tissue, plant tissue, an inanimate object, soil, a fluid (gas or liquid), genetic material, or a microbe. The sample may be a bodily fluid or a sample derived from a user's body, or the sample may be a non-bodily fluid or not derived from a user's body. The sample may be substantially stationary whereby the sample is not moving relative to the sensor, or the sample may be flowing whereby the sample is moving relative to the sensor. In some embodiments, the sensing assembly is not used to detect an analyte and is instead used to detect an absence of an analyte or used to detect the presence or absence of some other feature.

The non-user wearable sensing assembly can be configured for use in any desired application. Example applications include, but are not limited to, industrial processes, scientific instruments, sensing characteristics of trees, sensing a characteristic(s) of rocks, mineral exploration, underground water detection, and many other applications.

The techniques described herein can be used to detect the analyte presence, as well an amount of the analyte or a concentration of the analyte. The techniques described herein can be used to detect a single analyte or more than one analyte. Examples of the analyte(s) detected by the user wearable sensing assembly and the non-user wearable sensing assembly can include, but are not limited to, one or more of blood glucose, blood cholesterol, blood alcohol, white blood cells, or luteinizing hormone.

In one embodiment, a sensing system can include a user wearable sensing assembly that includes a sensor that is configured to detect an analyte in a user when the user wearable sensing assembly is worn by the user. The sensor includes at least one transmit antenna and at least one receive antenna. The at least one transmit antenna is positioned and arranged to transmit a signal into the user's body, wherein the signal is in a radio or microwave frequency range of the electromagnetic spectrum, and the at least one receive antenna is positioned and arranged to detect a response resulting from transmission of the signal by the at least one transmit antenna into the user's body. The system further includes a non-user wearable sensing assembly separate from the user wearable sensing assembly, where the non-user wearable sensing assembly includes a mounting location that is configured to permit removable mounting of the sensor to the non-user wearable sensing assembly so that the non-user wearable sensing assembly can perform a detection function using the sensor.

In another embodiment, a sensing system can include an in vivo sensing assembly that is configured to be worn by a user, where the in vivo sensing assembly includes a sensor portion that is removable from the in vivo sensing assembly and the sensor portion is configured to detect an analyte in the user when the in vivo sensing assembly is worn by the user. The sensor portion includes at least one transmit element and at least one receive element. The at least one transmit element is positioned and arranged to transmit a signal into the user's body, wherein the signal is in a radio or microwave frequency range of the electromagnetic spectrum, and the at least one receive element is positioned and arranged to detect a response resulting from transmission of the signal by the at least one transmit element into the user's body. The system can further include an in vitro sensing assembly separate from the in vivo sensing assembly, where the in vitro sensing assembly includes a mounting location that is configured to permit removable mounting of the sensor portion to the in vitro sensing assembly so that the in vitro sensing assembly can perform a detection function using the sensor portion.

In still another embodiment, a sensing method can include using a user wearable sensing assembly that includes a sensor removably mounted thereon to detect an analyte in a user when the user wearable sensing assembly is worn by the user, wherein the sensor includes at least one transmit antenna and at least one receive antenna, the at least one transmit antenna is positioned and arranged to transmit a signal into the user's body, wherein the signal is in a radio or microwave frequency range of the electromagnetic spectrum, and the at least one receive antenna is positioned and arranged to detect a response resulting from transmission of the signal by the at least one transmit antenna into the user's body. The sensor can be removed from the user wearable sensing assembly and installed at a mounting location of a non-user wearable sensing assembly that is configured to permit removable mounting of the sensor thereon. Thereafter, the non-user wearable sensing assembly can perform a detection function using the sensor installed on the non-user wearable sensing assembly.

In another embodiment, a sensing method can include using an in vivo sensing assembly that includes a sensor removably mounted thereon to detect an analyte in a user when the in vivo sensing assembly is worn by the user, wherein the sensor includes at least one transmit element and at least one receive element, the at least one transmit element is positioned and arranged to transmit a signal into the user's body, wherein the signal is in a radio or microwave frequency range of the electromagnetic spectrum, and the at least one receive element is positioned and arranged to detect a response resulting from transmission of the signal by the at least one transmit element into the user's body. The sensor can be removed from the in vivo sensing assembly and removably installed at a mounting location of an in vitro sensing assembly. Thereafter, the in vitro sensing assembly can perform a detection function using the sensor installed on the in vitro sensing assembly.

### DRAWINGS

FIG. 1 is a schematic depiction of a sensing system described herein.

FIG. 2 is a perspective view of an embodiment of a user wearable sensing assembly of the sensing system.

FIG. 3 is an exploded view showing the sensor of FIG. 2 removed from the strap.

FIG. 4 is a perspective view of an embodiment of a non-user wearable sensing assembly of the sensing system.

FIG. 5 is a top view of the non-user wearable sensing assembly depicting an example positioning of the sensor relative to a sample chamber.

FIG. 6 depicts an example of a sensing method described herein.

FIG. 7 is a schematic depiction of an example of a sensor that can be used.

FIGS. 8A-C illustrate different example orientations of antenna arrays that can be used in the sensor described herein.

FIGS. 9A-9I illustrate different examples of transmit and receive antennas with different geometries.

FIGS. 10A, 10B, 10C and 10D illustrate additional examples of different shapes that the ends of the transmit and receive antennas can have.

FIG. 11 is a schematic depiction of a sensor according to an embodiment.

### DETAILED DESCRIPTION

As used throughout this specification including the claims, the term “in vivo” is intended to refer to detecting an analyte within the body of a human or animal. As used throughout this specification including the claims, the term “in vitro” is intended to refer to detection that occurs outside the body of a human or animal.

A “user wearable sensing assembly” refers to a sensing assembly that is specifically configured to be worn by a human or animal during its regular and intended use to detect an analyte within (i.e. in vivo) the body of the human or animal. A “non-user wearable sensing assembly” refers to a sensing assembly that is intended to not be worn by a human or animal during its regular and intended use to perform a detection function outside (i.e. in vitro) the body of a human or animal.

For purposes of describing the concepts herein, the non-user wearable sensing assembly will be described in the examples below as being used to detect an analyte in a sample. In some embodiments, the user wearable and non-user wearable sensing assemblies are not used to detect an analyte and are instead used to detect an absence of an analyte or used to detect some other feature. However, the sensor described herein can be used with any type or configuration of non-user wearable sensing assembly. For example, the non-user wearable sensing assembly can be configured for use in an industrial process, configured for mounting on or adjacent to scientific instruments, configured for mounting on or adjacent to a tree to sense a characteristic(s) of the tree, configured for being mounted on or adjacent a rock to sense a characteristic(s) of the rock, configured for mounting on or adjacent to the ground for mineral exploration or underground water detection, and other configurations.

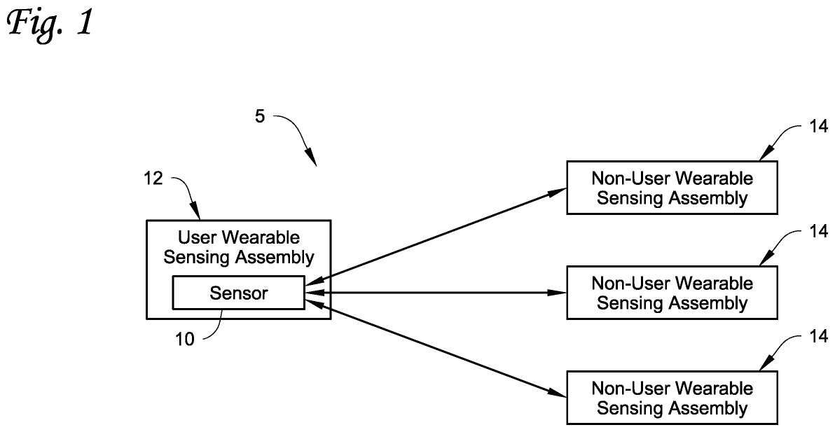

With reference to FIG. 1, an example of a sensing system 5 is illustrated. The system 5 includes an interchangeable sensor 10, a user wearable sensing assembly 12, and one or more non-user wearable sensing assemblies 14. The sensor 10 can detect a feature, such as an analyte, via spectroscopic techniques using non-optical frequencies such as in the radio or microwave frequency bands of the electromagnetic spectrum. The interchangeable sensor 10 is removably disposable on the user wearable sensing assembly 12 (also referred to as an in vivo sensing assembly) that can be worn by a user for non-invasively detecting an analyte in the user. The sensor 10 can be removed from the user wearable sensing assembly 12 and removably installed on the non-user wearable sensing assembly 14 (also referred to as an in vitro sensing assembly), each one of which can detect an analyte in a sample material using the sensor 10 mounted on the non-user wearable sensing assembly 14. In another embodiment, the entire user wearable sensing assembly 12 with the sensor 10 mounted thereon can be removably installed on any one of the non-user wearable sensing assemblies 14.

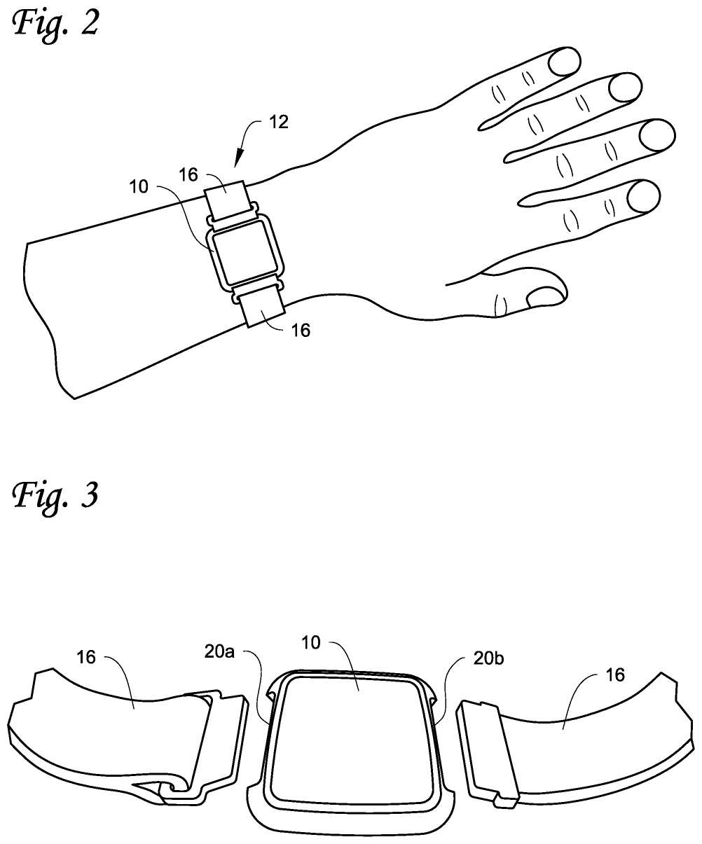

The user wearable sensing assembly 12 can be configured to be worn at any location on the user. In one non-limiting example illustrated in FIG. 2, the user wearable sensing assembly 12 can be configured to be worn on the user's arm, for example the user's wrist, with a wrist strap 16 that is directly or indirectly detachably fastenable to the sensor 10. In some embodiments, the user wearable sensing assembly 12 can be worn by an animal to detect an analyte within the animal.

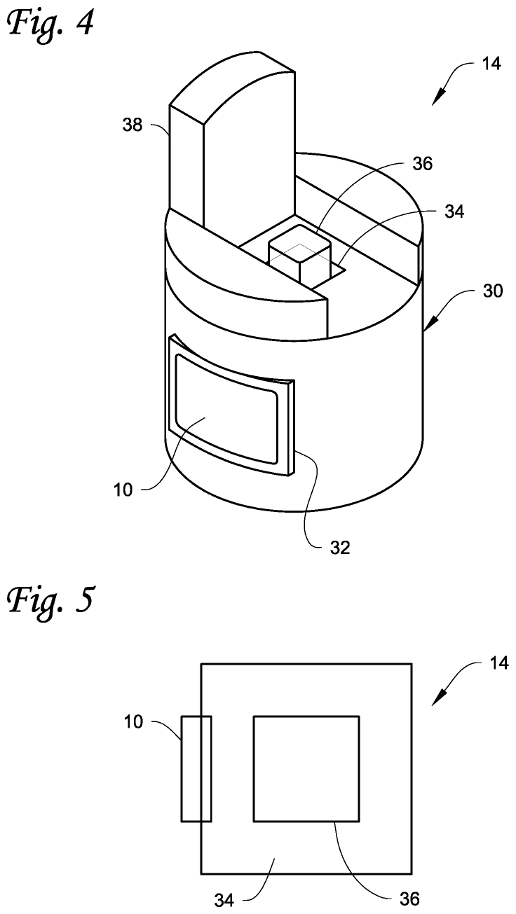

Returning to FIG. 1, each one of the non-user wearable sensing assemblies 14 can be any sensing assembly that is not user wearable and that is used to detect an analyte in a sample that is separate from the user. As discussed in further detail below in FIG. 4, each non-user wearable sensing assembly 14 includes a mounting location that is configured to permit removable mounting of the sensor 10 (or of the entire user wearable sensing assembly 12 including the sensor 10 and the wrist strap 16) to the non-user wearable sensing assembly 14 so that the non-user wearable sensing assembly 14 can detect an analyte using the sensor 10.

The analyte detected by the non-user wearable sensing assembly 14 can be a different analyte than the analyte detected by the user wearable sensing assembly 12. In another embodiment, the analyte detected by the non-user wearable sensing assembly 14 can be the same analyte that is detected by the user wearable sensing assembly 12. The sample used with the non-user wearable sensing assembly 14 can be a liquid, a gas, a solid, a semi-fluid, a semi-solid, a gel, and combinations thereof; human or non-human, animal or non-animal; biological or non-biological; or any other material that contains, or may contain, the analyte(s) that one may wish to detect. Examples of samples include, but are not limited to, human tissue, animal tissue, plant tissue, an inanimate object, soil, a fluid (gas or liquid), genetic material, or a microbe. The sample may be a bodily fluid or a sample derived from a user's body, or the sample may be a non-bodily fluid or not derived from a user's body. The sample may be substantially stationary whereby the sample is not moving relative to the sensor during detection, or the sample may be flowing whereby the sample is moving relative to the sensor during detection.

FIG. 2 illustrates an embodiment of the user wearable sensing assembly 12 in the form of a wrist worn assembly that is intended to be worn on the user's arm, for example around or near the user's wrist. In this embodiment, the sensor 10 is fastened to the wrist strap 16 which is used to fasten the assembly 12 to the user's arm. The wrist strap 16 can have any construction suitable for securing the sensor 10 to the user's arm. For example, the wrist strap 16 can have free ends that are secured to one another using a clasp, buckle, or the like; the wrist strap 16 can be a closed loop; the wrist strap 16 can be a single strap that doubles back onto itself to secure the assembly 12 to the user's arm; or the like. The wrist strap 16 can be flexible or rigid. The wrist strap 16 can be made of any suitable material including, but not limited to, rubber, plastic, steel, metal, leather, fabric material such as hook and loop fastener material, Nylon, wood, ceramic, and any other materials known for forming wrist straps for watches.

With reference to FIG. 3, the sensor 10 may be detachably connected to the wrist strap 16 in any manner that allows the wrist strap 16 to be detached from the sensor 10. Assuming that the wrist strap 16 is a two piece strap as depicted in FIG. 3, an end of each strap piece can be detachably connected to the sensor 10. The sensor 10 can include opposite slots 20a, 20b that receive the ends of the strap pieces. The detachable connections between the ends of the strap pieces and the sensor 10 can be achieved using the type of connection mechanisms used to connect watch straps to watch housings, for example using one or more spring bars. In other embodiments, the wrist strap 16 need not be detachable. Instead, the sensor 10 can be detachably fixed to a frame (not shown) that is fixed (detachably or non-detachably) to the wrist strap 16. In such an embodiment, the sensor 10 may be detached from the frame, leaving behind the frame fixed to the wrist strap 16.

In some embodiments, detachable connection of the sensor 10 to the wrist strap 16 is not required. Rather, the sensor 10 and the wrist strap 16 (i.e. the entire user wearable sensing assembly 12) can be detachably mounted to the non-user wearable sensing assembly 14. This embodiment would not require detaching of the sensor 10 from the wrist strap 16 in order to utilize the sensor 10 on the non-user wearable sensing assembly 14.

Referring to FIG. 4, an example of the non-user wearable sensing assembly 14 is illustrated. In this example, the non-user wearable sensing assembly 14 includes a sensor housing 30 that includes a mounting location 32 that is configured to permit removable mounting of the sensor 10 (or the entire assembly 12) to the non-user wearable sensing assembly 14 so that the non-user wearable sensing assembly 14 can detect an analyte using the sensor 10. The sensor 10 can be detachably secured to the mounting location 32 in any suitable manner that secures to the sensor 10 in suitable position to perform its sensing functions and permits detaching of the sensor 10 from the mounting location 32. For example, the sensor 10 can be mounted to the mounting location 32 using connection mechanisms similar to the connection mechanisms used to connect the wrist strap 16 and the sensor 10. In other embodiments, the sensor 10 can be secured in the mounting location 32 via a friction fit, using one or more magnets, or other types of securement.

The sensor housing 30 can further include a sample chamber 34 that is configured to receive a sample. In the illustrated example, the sample chamber 34 can receive a container 36 at least partially therein that is configured to contain the sample during a test. A lid 38 may close the sample chamber 34. The sample chamber 34 and the container 36 can have any configurations suitable for permitting a sample held in the container 36 to be tested. The sample chamber 34 holds the container 36 during a test. The container 36 has a configuration that is suitable for containing a sample during operation of the sensor 10 and that permits travel of electromagnetic waves that are in the radio or microwave frequency bands of the electromagnetic spectrum through at least one wall thereof into and from the sample. In one embodiment, the container 36 can be a cuvette made of glass or plastic. The sample chamber 34 can be square, rectangular, round, triangular or other shape in cross-section. The container 36 can be square, rectangular, round, triangular or other shape in cross-section.

FIG. 5 illustrates an example where the sample chamber 34 and the container 36 are each generally square or rectangular in cross-section. Many other shapes and combinations of shapes for the sample chamber 14 and the container 18 are possible. The mounting location 32 is adjacent to the sample chamber 34; and when the sensor 10 is mounted to the mounting location 32, the sensor 10 is properly positioned to detect an analyte in a material that is contained in the container 36. As described in further detail below, at least one transmit antenna and at least one receive antenna of the sensor face the sample chamber 34 and the container 36 when the sensor 10 is properly mounted in the mounting location 32.

The sample used with the non-user wearable sensing assembly 14 can be stationary or flowing. The sample can be a liquid, gas, vapor, solid, semi-solid, gel, and combinations thereof.

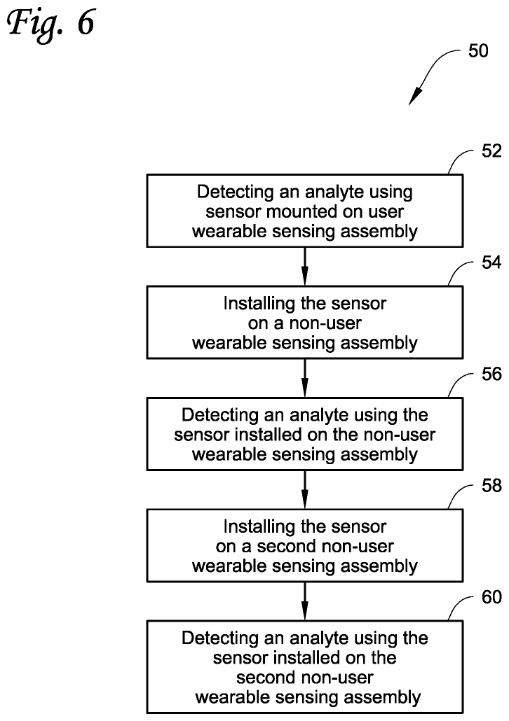

Referring to FIG. 6 together with FIG. 1, a sensing method 50 that can be implemented using the system 5 is illustrated. In this example, the method 50 includes a step 52 of using the user wearable sensing assembly 12 with the sensor 10 mounted thereon to detect an analyte in the user that is wearing the user wearable sensing assembly 12. Thereafter, in a step 54, the sensor 10 (either separate from the wrist strap 16 or while attached to the wrist strap 16) is installed on the non-user wearable sensing assembly 14 at the mounting location thereof. The non-user wearable sensing assembly 14 is then used at step 56 to detect an analyte in a material that is in the sample chamber thereof. If the sensor 10 has been removed from the wrist strap 16, the sensor 10 may then be reinstalled on the wrist strap 16. Optionally, the sensor 10 may be removed from the non-user wearable sensing assembly 14 and then installed in a second non-user wearable sensing assembly 14 in step 58 which is then used to detect an analyte in a material that is in the sample chamber thereof in step 60.

The sensor 10 described herein can be configured in any way that allows the sensor 10 to perform its sensing functions described herein via spectroscopic techniques using non-optical frequencies such as in the radio or microwave frequency bands of the electromagnetic spectrum. In general, the sensor 10 includes at least one transmit antenna (which may also be referred to as a transmit element) that functions to transmit a generated transmit signal that is in a radio or microwave frequency range of the electromagnetic spectrum, and at least one receive antenna (which may also be referred to as a receive element) that functions to detect a response resulting from transmission of the transmit signal by the transmit antenna into the user's body or into the sample contained in the sample chamber. In some embodiments, the transmit antenna and the receive antenna are decoupled from one another which improves the detection performance of the sensor 10.

In one embodiment, the sensor 10 can have a construction like the sensors disclosed in U.S. Pat. No. 10,548,503 which is incorporated herein by reference in its entirety. In another embodiment, the sensor 10 can have a construction like the sensors disclosed in U.S. Patent Application Publication 2019/0008422. In another embodiment, the sensor 10 can have a construction like the sensors disclosed in U.S. Patent Application Publication 2020/0187791.

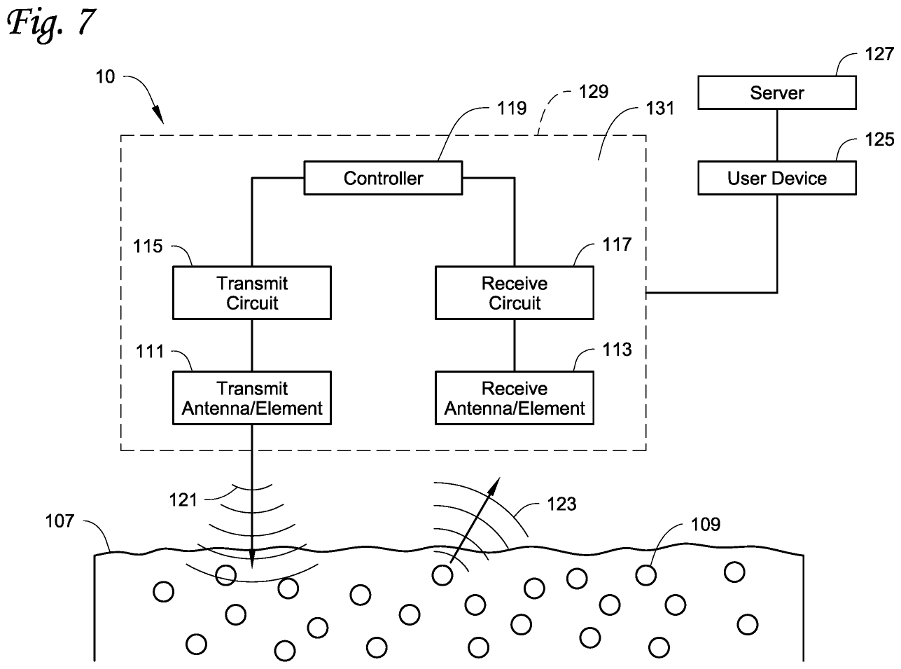

FIGS. 7-11 illustrate details of an example of the sensor 10 that can be used. Further details on the sensor in FIGS. 7-11 can be found in pending U.S. Patent Application 62/951,756 filed on Dec. 20, 2019 and entitled Non-Invasive Analyte Sensor And System With Decoupled Transmit And Receive Antennas, and in pending U.S. Patent Application 62/971,053 filed on Feb. 6, 2020 and entitled Non-Invasive Detection Of An Analyte Using Different Combinations of Antennas That Can Transmit Or Receive, the entire contents of both applications are incorporated herein by reference.

In the sensor 10, the transmit antenna transmits a signal, which has at least two frequencies in the radio or microwave frequency range, toward and into the wearer's arm or into the sample in the sample chamber (each of which can be referred to as a “target”). The signal with the at least two frequencies can be formed by separate signal portions, each having a discrete frequency, that are transmitted separately at separate times at each frequency. In another embodiment, the signal with the at least two frequencies may be part of a complex signal that includes a plurality of frequencies including the at least two frequencies. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The receive antenna detects a response resulting from transmission of the signal by the transmit antenna into the target containing the analyte.

The transmit antenna and the receive antenna are decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

The signal(s) detected by the receive antenna can be analyzed to detect the analyte based on the intensity of the received signal(s) and reductions in intensity at one or more frequencies where the analyte absorbs the transmitted signal. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e. without demultiplexing the complex signal) to detect the analyte as long as the detected signal provides enough information to make the analyte detection. In addition, the signal(s) detected by the receive antenna can be separate signal portions, each having a discrete frequency.

In one embodiment, the sensor 10 can be used to detect the presence of at least one analyte in the target. In another embodiment, the sensor can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, a fluid, for example blood, interstitial fluid, cerebral spinal fluid, lymph fluid or urine, human tissue, animal tissue, plant tissue, an inanimate object, soil, genetic material, or a microbe.

The analyte(s) can be any analyte that one may wish to detect. The analyte can be human or non-human, animal or non-animal, biological or non-biological. For example, the analyte(s) can include, but is not limited to, one or more of blood glucose, blood alcohol, white blood cells, or luteinizing hormone. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) that are detected can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 143 hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

Referring now to FIG. 7, an embodiment of the sensor 10 is illustrated. The sensor 10 is depicted relative to a target 107 (which can be the wearer's arm or a sample contained in the sample chamber of FIG. 4) that contains an analyte of interest 109. In this example, the sensor 10 is depicted as including an antenna array that includes a transmit antenna/element 111 (hereinafter “transmit antenna 111”) and a receive antenna/element 113 (hereinafter “receive antenna 113”). The sensor 10 further includes a transmit circuit 115, a receive circuit 117, and a controller 119. As discussed further below, the sensor 10 can also include a power supply, such as a battery (not shown in FIG. 7).

The transmit antenna 111 is positioned, arranged and configured to transmit a signal 121 that is the radio frequency (RF) or microwave range of the electromagnetic spectrum into the target 107. The transmit antenna 111 can be an electrode or any other suitable transmitter of electromagnetic signals in the radio frequency (RF) or microwave range. The transmit antenna 111 can have any arrangement and orientation relative to the target 107 that is sufficient to allow the analyte sensing to take place. In one non-limiting embodiment, the transmit antenna 111 can be arranged to face in a direction that is substantially toward the target 107.

The signal 121 transmitted by the transmit antenna 111 is generated by the transmit circuit 115 which is electrically connectable to the transmit antenna 111. The transmit circuit 115 can have any configuration that is suitable to generate a transmit signal to be transmitted by the transmit antenna 111. Transmit circuits for generating transmit signals in the RF or microwave frequency range are well known in the art. In one embodiment, the transmit circuit 115 can include, for example, a connection to a power source, a frequency generator, and optionally filters, amplifiers or any other suitable elements for a circuit generating an RF or microwave frequency electromagnetic signal. In an embodiment, the signal generated by the transmit circuit 115 can have at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit circuit 115 can be configured to sweep through a range of frequencies that are within the range of about 10 kHz to about 100 GHz, or in another embodiment a range of about 300 MHz to about 6000 MHz. In an embodiment, the transmit circuit 115 can be configured to produce a complex transmit signal, the complex signal including a plurality of signal components, each of the signal components having a different frequency. The complex signal can be generated by blending or multiplexing multiple signals together followed by transmitting the complex signal whereby the plurality of frequencies are transmitted at the same time.

The receive antenna 113 is positioned, arranged, and configured to detect one or more electromagnetic response signals 123 that result from the transmission of the transmit signal 121 by the transmit antenna 111 into the target 107 and impinging on the analyte 109. The receive antenna 113 can be an electrode or any other suitable receiver of electromagnetic signals in the radio frequency (RF) or microwave range. In an embodiment, the receive antenna 113 is configured to detect electromagnetic signals having at least two frequencies, each of which is in the range from about 10 kHz to about 100 GHz, or in another embodiment a range from about 300 MHz to about 6000 MHz. The receive antenna 113 can have any arrangement and orientation relative to the target 107 that is sufficient to allow detection of the response signal(s) 123 to allow the analyte sensing to take place. In one non-limiting embodiment, the receive antenna 113 can be arranged to face in a direction that is substantially toward the target 107.

The receive circuit 117 is electrically connectable to the receive antenna 113 and conveys the received response from the receive antenna 113 to the controller 119. The receive circuit 117 can have any configuration that is suitable for interfacing with the receive antenna 113 to convert the electromagnetic energy detected by the receive antenna 113 into one or more signals reflective of the response signal(s) 123. The construction of receive circuits are well known in the art. The receive circuit 117 can be configured to condition the signal(s) prior to providing the signal(s) to the controller 119, for example through amplifying the signal(s), filtering the signal(s), or the like. Accordingly, the receive circuit 117 may include filters, amplifiers, or any other suitable components for conditioning the signal(s) provided to the controller 119. In an embodiment, at least one of the receive circuit 117 or the controller 119 can be configured to decompose or demultiplex a complex signal, detected by the receive antenna 113, including a plurality of signal components each at different frequencies into each of the constituent signal components. In an embodiment, decomposing the complex signal can include applying a Fourier transform to the detected complex signal. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection.

The controller 119 controls the operation of the sensor 10. The controller 119, for example, can direct the transmit circuit 115 to generate a transmit signal to be transmitted by the transmit antenna 111. The controller 119 further receives signals from the receive circuit 117. The controller 119 can optionally process the signals from the receive circuit 117 to detect the analyte(s) 109 in the target 107. In one embodiment, the controller 119 may optionally be in communication with at least one external device 125 such as a user device and/or a remote server 127, for example through one or more wireless connections such as Bluetooth, wireless data connections such a 4G, 5G, LTE or the like, or Wi-Fi. If provided, the external device 125 and/or remote server 127 may process (or further process) the signals that the controller 119 receives from the receive circuit 117, for example to detect the analyte(s) 109. If provided, the external device 125 may be used to provide communication between the sensor 10 and the remote server 127, for example using a wired data connection or via a wireless data connection or Wi-Fi of the external device 125 to provide the connection to the remote server 127.

With continued reference to FIG. 7, the sensor 10 may include a sensor housing 129 (shown in dashed lines) that defines an interior space 131. Components of the sensor 10 may be attached to and/or disposed within the housing 129. For example, the transmit antenna 111 and the receive antenna 113 are attached to the housing 129. In some embodiments, the antennas 111, 113 may be entirely or partially within the interior space 131 of the housing 129. In some embodiments, the antennas 111, 113 may be attached to the housing 129 but at least partially or fully located outside the interior space 131. In some embodiments, the transmit circuit 115, the receive circuit 117 and the controller 119 are attached to the housing 129 and disposed entirely within the sensor housing 129.

The receive antenna 113 is decoupled or detuned with respect to the transmit antenna 111 such that electromagnetic coupling between the transmit antenna 111 and the receive antenna 113 is reduced. The decoupling of the transmit antenna 111 and the receive antenna 113 increases the portion of the signal(s) detected by the receive antenna 113 that is the response signal(s) 123 from the target 107, and minimizes direct receipt of the transmitted signal 121 by the receive antenna 113. The decoupling of the transmit antenna 111 and the receive antenna 113 results in transmission from the transmit antenna 111 to the receive antenna 113 having a reduced forward gain (S₂₁) and an increased reflection at output (S22) compared to antenna systems having coupled transmit and receive antennas.

In an embodiment, coupling between the transmit antenna 111 and the receive antenna 113 is 95% or less. In another embodiment, coupling between the transmit antenna 111 and the receive antenna 113 is 90% or less. In another embodiment, coupling between the transmit antenna 111 and the receive antenna 113 is 85% or less. In another embodiment, coupling between the transmit antenna 111 and the receive antenna 113 is 75% or less.

Any technique for reducing coupling between the transmit antenna 111 and the receive antenna 113 can be used. For example, the decoupling between the transmit antenna 111 and the receive antenna 113 can be achieved by one or more intentionally fabricated configurations and/or arrangements between the transmit antenna 111 and the receive antenna 113 that is sufficient to decouple the transmit antenna 111 and the receive antenna 113 from one another.

For example, in one embodiment described further below, the decoupling of the transmit antenna 111 and the receive antenna 113 can be achieved by intentionally configuring the transmit antenna 111 and the receive antenna 113 to have different geometries from one another. Intentionally different geometries refers to different geometric configurations of the transmit and receive antennas 111, 113 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 111 and the receive antenna 113 is to provide appropriate spacing between each antenna 111, 113 that is sufficient to decouple the antennas 111, 113 and force a proportion of the electromagnetic lines of force of the transmitted signal 121 into the target 107 thereby minimizing or eliminating as much as possible direct receipt of electromagnetic energy by the receive antenna 113 directly from the transmit antenna 111 without traveling into the target 107. The appropriate spacing between each antenna 111, 113 can be determined based upon factors that include, but are not limited to, the output power of the signal from the transmit antenna 111, the size of the antennas 111, 113, the frequency or frequencies of the transmitted signal, and the presence of any shielding between the antennas. This technique helps to ensure that the response detected by the receive antenna 113 is measuring the analyte 109 and is not just the transmitted signal 121 flowing directly from the transmit antenna 111 to the receive antenna 113. In some embodiments, the appropriate spacing between the antennas 111, 113 can be used together with the intentional difference in geometries of the antennas 111, 113 to achieve decoupling.

In one embodiment, the transmit signal that is transmitted by the transmit antenna 111 can have at least two different frequencies, for example upwards of 7 to 12 different and discrete frequencies. In another embodiment, the transmit signal can be a series of discrete, separate signals with each separate signal having a single frequency or multiple different frequencies.

In one embodiment, the transmit signal (or each of the transmit signals) can be transmitted over a transmit time that is less than, equal to, or greater than about 300 ms. In another embodiment, the transmit time can be than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%.

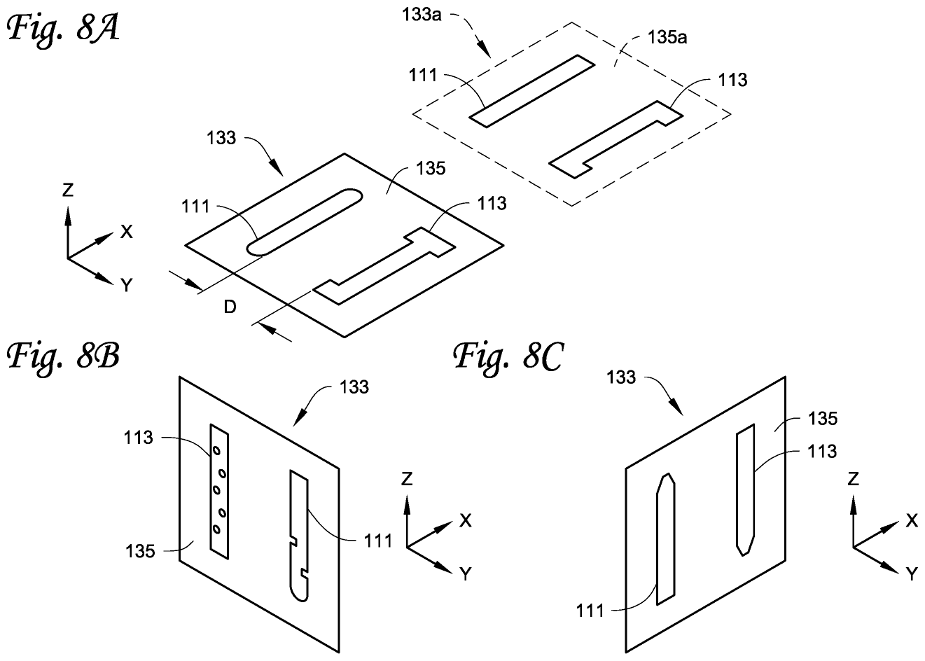

FIGS. 8A-8C illustrate examples of antenna arrays 133 that can be used in the sensor 10 and how the antenna arrays 133 can be oriented. Many orientations of the antenna arrays 133 are possible, and any orientation can be used as long as the sensor 10 can perform its primary function of sensing the analyte 109.

In FIG. 8A, the antenna array 133 includes the transmit antenna 111 and the receive antenna 113 disposed on a substrate 135 which may be substantially planar. This example depicts the array 133 disposed substantially in an X-Y plane. In this example, dimensions of the antennas 111, 113 in the X and Y-axis directions can be considered lateral dimensions, while a dimension of the antennas 111, 113 in the Z-axis direction can be considered a thickness dimension. In this example, each of the antennas 111, 113 has at least one lateral dimension (measured in the X-axis direction and/or in the Y-axis direction) that is greater than the thickness dimension thereof (in the Z-axis direction). In other words, the transmit antenna 111 and the receive antenna 113 are each relatively flat or of relatively small thickness in the Z-axis direction compared to at least one other lateral dimension measured in the X-axis direction and/or in the Y-axis direction.

In use of the embodiment in FIG. 8A, the sensor and the array 133 may be positioned relative to the target 107 such that the target 107 is below the array 133 in the Z-axis direction or above the array 133 in the Z-axis direction whereby one of the faces of the antennas 111, 113 face toward the target 107. Alternatively, the target 107 can be positioned to the left or right sides of the array 133 in the X-axis direction whereby one of the ends of each one of the antennas 111, 113 face toward the target 107. Alternatively, the target 107 can be positioned to the sides of the array 133 in the Y-axis direction whereby one of the sides of each one of the antennas 111, 113 face toward the target 107.

The sensor 10 can also be provided with one or more additional antenna arrays in addition the antenna array 133. For example, FIG. 8A also depicts an optional second antenna array 133a that includes the transmit antenna 111 and the receive antenna 113 disposed on a substrate 135a which may be substantially planar. Like the array 133, the array 133a may also be disposed substantially in the X-Y plane, with the arrays 133, 133a spaced from one another in the X-axis direction.

In FIG. 8B, the antenna array 133 is depicted as being disposed substantially in the Y-Z plane. In this example, dimensions of the antennas 111, 113 in the Y and Z-axis directions can be considered lateral dimensions, while a dimension of the antennas 111, 113 in the X-axis direction can be considered a thickness dimension. In this example, each of the antennas 111, 113 has at least one lateral dimension (measured in the Y-axis direction and/or in the Z-axis direction) that is greater than the thickness dimension thereof (in the X-axis direction). In other words, the transmit antenna 111 and the receive antenna 113 are each relatively flat or of relatively small thickness in the X-axis direction compared to at least one other lateral dimension measured in the Y-axis direction and/or in the Z-axis direction.

In use of the embodiment in FIG. 8B, the sensor and the array 133 may be positioned relative to the target 107 such that the target 107 is below the array 133 in the Z-axis direction or above the array 133 in the Z-axis direction whereby one of the ends of each one of the antennas 111, 113 face toward the target 107. Alternatively, the target 107 can be positioned in front of or behind the array 133 in the X-axis direction whereby one of the faces of each one of the antennas 111, 113 face toward the target 107. Alternatively, the target 107 can be positioned to one of the sides of the array 133 in the Y-axis direction whereby one of the sides of each one of the antennas 111, 113 face toward the target 107.

In FIG. 8C, the antenna array 133 is depicted as being disposed substantially in the X-Z plane. In this example, dimensions of the antennas 111, 113 in the X and Z-axis directions can be considered lateral dimensions, while a dimension of the antennas 111, 113 in the Y-axis direction can be considered a thickness dimension. In this example, each of the antennas 111, 113 has at least one lateral dimension (measured in the X-axis direction and/or in the Z-axis direction) that is greater than the thickness dimension thereof (in the Y-axis direction). In other words, the transmit antenna 111 and the receive antenna 113 are each relatively flat or of relatively small thickness in the Y-axis direction compared to at least one other lateral dimension measured in the X-axis direction and/or in the Z-axis direction.

In use of the embodiment in FIG. 8C, the sensor and the array 133 may be positioned relative to the target 107 such that the target 107 is below the array 133 in the Z-axis direction or above the array 133 in the Z-axis direction whereby one of the ends of each one of the antennas 111, 113 face toward the target 107. Alternatively, the target 107 can be positioned to the left or right sides of the array 133 in the X-axis direction whereby one of the sides of each one of the antennas 111, 113 face toward the target 107. Alternatively, the target 107 can be positioned in front of or in back of the array 133 in the Y-axis direction whereby one of the faces of each one of the antennas 111, 113 face toward the target 107.

The arrays 133, 133a in FIGS. 8A-8C need not be oriented entirely within a plane such as the X-Y plane, the Y-Z plane or the X-Z plane. Instead, the arrays 133, 133a can be disposed at angles to the X-Y plane, the Y-Z plane and the X-Z plane.

Decoupling Antennas Using Differences in Antenna Geometries

As mentioned above, one technique for decoupling the transmit antenna 111 from the receive antenna 113 is to intentionally configure the transmit antenna 111 and the receive antenna 113 to have intentionally different geometries. Intentionally different geometries refers to differences in geometric configurations of the transmit and receive antennas 111, 113 that are intentional, and is distinct from differences in geometry of the transmit and receive antennas 111, 113 that may occur by accident or unintentionally, for example due to manufacturing errors or tolerances when fabricating the antennas 111, 113.

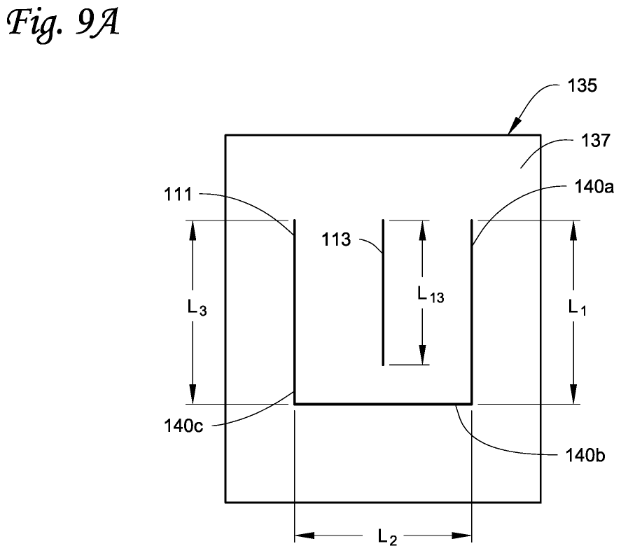

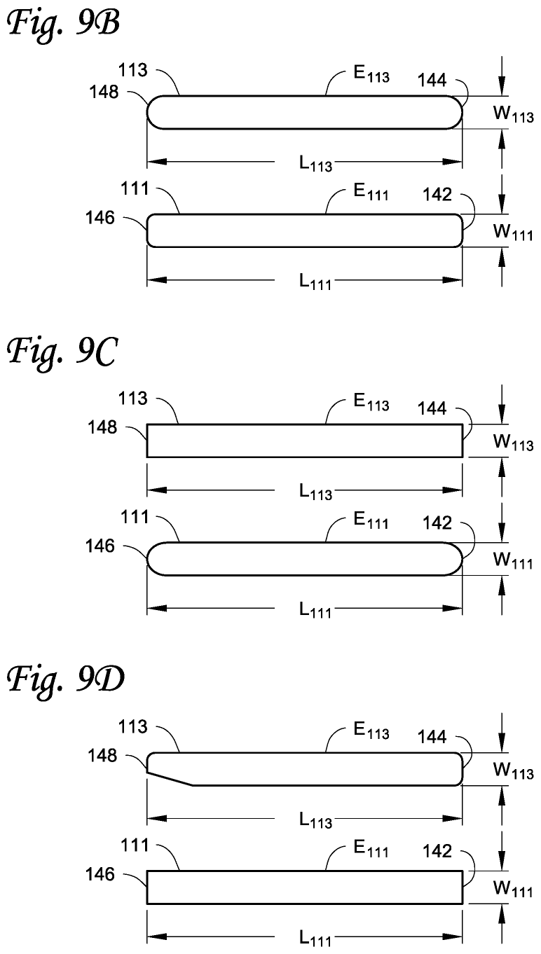

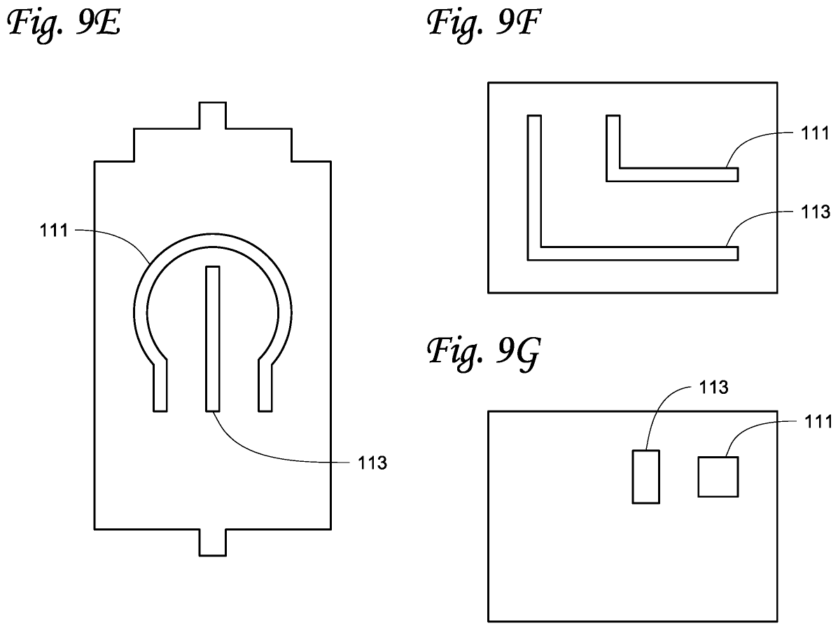

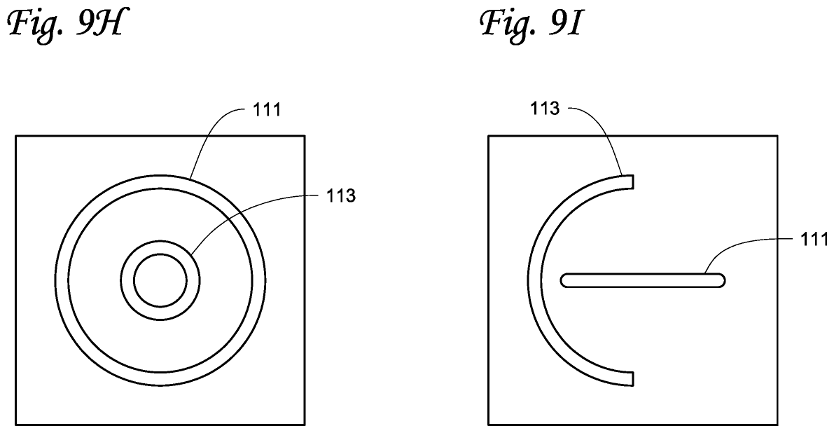

The different geometries of the antennas 111, 113 may manifest itself, and may be described, in a number of different ways. For example, in a plan view of each of the antennas 111, 113 (such as in FIGS. 9A-I), the shapes of the perimeter edges of the antennas 111, 113 may be different from one another. The different geometries may result in the antennas 111, 113 having different surface areas in plan view. The different geometries may result in the antennas 111, 113 having different aspect ratios in plan view (i.e. a ratio of their sizes in different dimensions; for example, as discussed in further detail below, the ratio of the length divided by the width of the antenna 111 may be different than the ratio of the length divided by the width for the antenna 113). In some embodiments, the different geometries may result in the antennas 111, 113 having any combination of different perimeter edge shapes in plan view, different surface areas in plan view, and/or different aspect ratios. In some embodiments, the antennas 111, 113 may have one or more holes formed therein (see FIG. 8B) within the perimeter edge boundary, or one or more notches formed in the perimeter edge (see FIG. 8B).

So as used herein, a difference in geometry or a difference in geometrical shape of the antennas 111, 113 refers to any intentional difference in the figure, length, width, size, shape, area closed by a boundary (i.e. the perimeter edge), etc. when the respective antenna 111, 113 is viewed in a plan view.

The antennas 111, 113 can have any configuration and can be formed from any suitable material that allows them to perform the functions of the antennas 111, 113 as described herein. In one embodiment, the antennas 111, 113 can be formed by strips of material. A strip of material can include a configuration where the strip has at least one lateral dimension thereof greater than a thickness dimension thereof when the antenna is viewed in a plan view (in other words, the strip is relatively flat or of relatively small thickness compared to at least one other lateral dimension, such as length or width when the antenna is viewed in a plan view as in FIGS. 9A-I). A strip of material can include a wire. The antennas 111, 113 can be formed from any suitable conductive material(s) including metals and conductive non-metallic materials. Examples of metals that can be used include, but are not limited to, copper or gold. Another example of a material that can be used is non-metallic materials that are doped with metallic material to make the non-metallic material conductive.

In FIGS. 8A-8C, the antennas 111, 113 within each one of the arrays 133, 133a have different geometries from one another. In addition, FIGS. 9A-I illustrate plan views of additional examples of the antennas 111, 113 having different geometries from one another. The examples in FIGS. 8A-8C and 9A-I are not exhaustive and many different configurations are possible.

With reference initially to FIG. 9A, a plan view of an antenna array having two antennas with different geometries is illustrated. In this example (as well as for the examples in FIGS. 8A-8C and 9B-9I), for sake of convenience in describing the concepts herein, one antenna is labeled as the transmit antenna 111 and the other antenna is labeled as the receive antenna 113. However, the antenna labeled as the transmit antenna 111 could be the receive antenna 113, while the antenna labeled as the receive antenna 113 could be the transmit antenna 111. Each of the antennas 111, 113 are disposed on the substrate 135 having a planar surface 137.

The antennas 111, 113 can be formed as linear strips or traces on the surface 137. In this example, the antenna 111 is generally U-shaped and has a first linear leg 140a, a second linear leg 140b that extends perpendicular to the first leg 140a, and a third linear leg 140c that extends parallel to the leg 140a. Likewise, the antenna 113 is formed by a single leg that extends parallel to, and between, the legs 140a, 140c.

In the example depicted in FIG. 9A, each one of the antennas 111, 113 has at least one lateral dimension that is greater than a thickness dimension thereof (in FIG. 9A, the thickness dimension would extend into/from the page when viewing FIG. 9A). For example, the leg 140a of the antenna 111 extends in one direction (i.e. a lateral dimension) an extent that is greater than a thickness dimension of the leg 140a extending into or out of the page; the leg 140b of the antenna 111 extends in a direction (i.e. a lateral dimension) an extent that is greater than a thickness dimension of the leg 140b extending into or out of the page; and the leg 140c of the antenna 111 extends in one direction (i.e. a lateral dimension) an extent that is greater than a thickness dimension of the leg 140c extending into or out of the page. Likewise, the antenna 113 extends in one direction (i.e. a lateral dimension) an extent that is greater than a thickness dimension of the antenna 13 extending into or out of the page.

The antennas 111, 113 also differ in geometry from one another in that the total linear length of the antenna 111 (determined by adding the individual lengths L₁, L₂, L₃ of the legs 140a-c together) when viewed in plan view is greater than the length L₁₃ of the antenna 13 when viewed in plan view.

FIG. 9B illustrates another plan view of an antenna array having two antennas with different geometries. In this example, the antennas 111, 113 are illustrated as substantially linear strips each with a lateral length L₁₁₁, L₁₁₃, a lateral width W₁₁₁, W₁₁₃, and a perimeter edge E₁₁₁, E₁₁₃. The perimeter edges E₁₁₁, E₁₁₃ extend around the entire periphery of the antennas 111, 113 and bound an area in plan view. In this example, the lateral length L₁₁₁, L₁₁₃ and/or the lateral width W₁₁₁, W₁₁₃ is greater than a thickness dimension of the antennas 111, 113 extending into/from the page when viewing FIG. 9B. In this example, the antennas 111, 113 differ in geometry from one another in that the shapes of the ends of the antennas 111, 113 differ from one another. For example, when viewing FIG. 9B, the right end 142 of the antenna 111 has a different shape than the right end 144 of the antenna 113. Similarly, the left end 146 of the antenna 111 may have a similar shape as the right end 142, but differs from the left end 148 of the antenna 113 which may have a similar shape as the right end 144. It is also possible that the lateral lengths L₁₁₁, L₁₁₃ and/or the lateral widths W₁₁₁, W₁₁₃ of the antennas 111, 113 could differ from one another.

FIG. 9C illustrates another plan view of an antenna array having two antennas with different geometries that is somewhat similar to FIG. 9B. In this example, the antennas 111, 113 are illustrated as substantially linear strips each with the lateral length L₁₁₁, L₁₁₃, the lateral width W₁₁₁, W₁₁₃, and the perimeter edge E₁₁₁, E₁₁₃. The perimeter edges E₁₁₁, E₁₁₃ extend around the entire periphery of the antennas 111, 113 and bound an area in plan view. In this example, the lateral length L₁₁₁, L₁₁₃ and/or the lateral width W₁₁₁, W₁₁₃ is greater than a thickness dimension of the antennas 111, 113 extending into/from the page when viewing FIG. 9C. In this example, the antennas 111, 113 differ in geometry from one another in that the shapes of the ends of the antennas 111, 113 differ from one another. For example, when viewing FIG. 9C, the right end 142 of the antenna 111 has a different shape than the right end 144 of the antenna 113. Similarly, the left end 146 of the antenna 111 may have a similar shape as the right end 142, but differs from the left end 148 of the antenna 113 which may have a similar shape as the right end 144. In addition, the lateral widths W₁₁₁, W₁₁₃ of the antennas 111, 113 differ from one another. It is also possible that the lateral lengths L₁₁₁, L₁₁₃ of the antennas 111, 113 could differ from one another.

FIG. 9D illustrates another plan view of an antenna array having two antennas with different geometries that is somewhat similar to FIGS. 9B and 9C. In this example, the antennas 111, 113 are illustrated as substantially linear strips each with the lateral length L₁₁₁, L₁₁₃, the lateral width W₁₁₁, W₁₁₃, and the perimeter edge E₁₁₁, E₁₁₃. The perimeter edges E₁₁₁, E₁₁₃ extend around the entire periphery of the antennas 111, 113 and bound an area in plan view. In this example, the lateral length L₁₁₁, L₁₁₃ and/or the lateral width W₁₁₁, W₁₁₃ is greater than a thickness dimension of the antennas 111, 113 extending into/from the page when viewing FIG. 9D. In this example, the antennas 111, 113 differ in geometry from one another in that the shapes of the ends of the antennas 111, 113 differ from one another. For example, when viewing FIG. 9D, the right end 142 of the antenna 111 has a different shape than the right end 144 of the antenna 113. Similarly, the left end 146 of the antenna 111 may have a similar shape as the right end 142, but differs from the left end 148 of the antenna 113 which may have a similar shape as the right end 144. In addition, the lateral widths W₁₁₁, W₁₁₃ of the antennas 111, 113 differ from one another. It is also possible that the lateral lengths L₁₁₁, L₁₁₃ of the antennas 111, 113 could differ from one another.

FIG. 9E illustrates another plan view of an antenna array having two antennas with different geometries on a substrate. In this example, the antenna 111 is illustrated as being a strip of material having a generally horseshoe shape, while the antenna 113 is illustrated as being a strip of material that is generally linear. The planar shapes (i.e. geometries) of the antennas 111, 113 differ from one another. In addition, the total length of the antenna 111 (measured from one end to the other) when viewed in plan view is greater than the length of the antenna 113 when viewed in plan.

FIG. 9F illustrates another plan view of an antenna array having two antennas with different geometries on a substrate. In this example, the antenna 111 is illustrated as being a strip of material forming a right angle, and the antenna 113 is also illustrated as being a strip of material that forms a larger right angle. The planar shapes (i.e. geometries) of the antennas 111, 113 differ from one another since the total area in plan view of the antenna 113 is greater than the total area in plan view of the antenna 111. In addition, the total length of the antenna 111 (measured from one end to the other) when viewed in plan view is less than the length of the antenna 113 when viewed in plan.

FIG. 9G illustrates another plan view of an antenna array having two antennas with different geometries on a substrate. In this example, the antenna 111 is illustrated as being a strip of material forming a square, and the antenna 113 is illustrated as being a strip of material that forms a rectangle. The planar shapes (i.e. geometries) of the antennas 111, 113 differ from one another. In addition, at least one of the width/length of the antenna 111 when viewed in plan view is less than one of the width/length of the antenna 113 when viewed in plan.

FIG. 9H illustrates another plan view of an antenna array having two antennas with different geometries on a substrate. In this example, the antenna 111 is illustrated as being a strip of material forming a circle when viewed in plan, and the antenna 113 is also illustrated as being a strip of material that forms a smaller circle when viewed in plan surrounded by the circle formed by the antenna 111. The planar shapes (i.e. geometries) of the antennas 111, 113 differ from one another due to the different sizes of the circles.

FIG. 9I illustrates another plan view of an antenna array having two antennas with different geometries on a substrate. In this example, the antenna 111 is illustrated as being a linear strip of material, and the antenna 113 is illustrated as being a strip of material that forms a semi-circle when viewed in plan. The planar shapes (i.e. geometries) of the antennas 111, 113 differ from one another due to the different shapes/geometries of the antennas 111, 113.

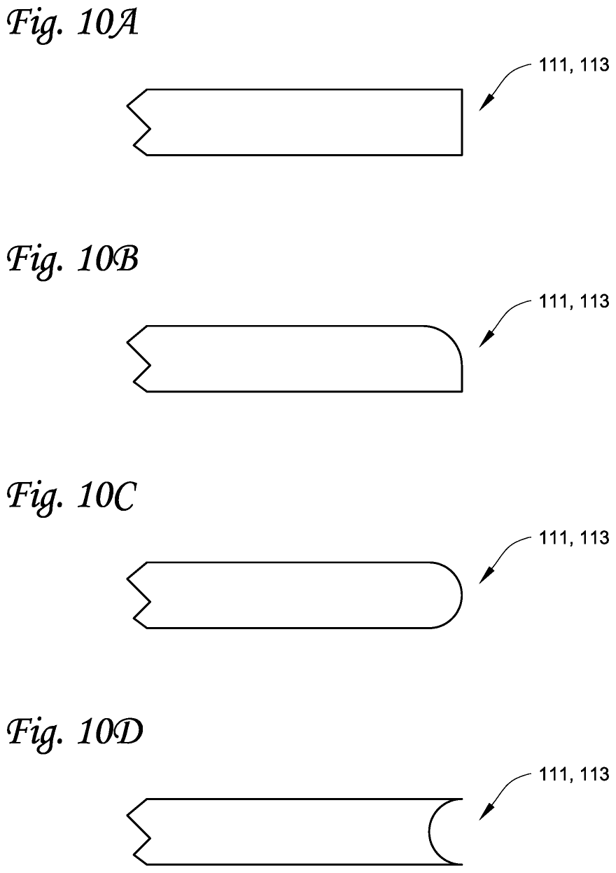

10A-D are plan views of additional examples of different shapes that the ends of the transmit and receive antennas 111, 113 can have to achieve differences in geometry. Either one of, or both of, the ends of the antennas 111, 113 can have the shapes in FIGS. 10A-D, including in the embodiments in FIGS. 9A-I. FIG. 10A depicts the end as being generally rectangular. FIG. 10B depicts the end as having one rounded corner while the other corner remains a right angle. FIG. 10C depicts the entire end as being rounded or outwardly convex. FIG. 10D depicts the end as being inwardly concave. Many other shapes are possible.

Another technique to achieve decoupling of the antennas 111, 113 is to use an appropriate spacing between each antenna 111, 113 with the spacing being sufficient to force most or all of the signal(s) transmitted by the transmit antenna 111 into the target, thereby minimizing the direct receipt of electromagnetic energy by the receive antenna 113 directly from the transmit antenna 111. The appropriate spacing can be used by itself to achieve decoupling of the antennas 111, 113. In another embodiment, the appropriate spacing can be used together with differences in geometry of the antennas 111, 113 to achieve decoupling.

Referring to FIG. 8A, there is a spacing D between the transmit antenna 111 and the receive antenna 113 at the location indicated. The spacing D between the antennas 111, 113 may be constant over the entire length (for example in the X-axis direction) of each antenna 111, 113, or the spacing D between the antennas 111, 113 could vary. Any spacing D can be used as long as the spacing D is sufficient to result in most or all of the signal(s) transmitted by the transmit antenna 111 reaching the target and minimizing the direct receipt of electromagnetic energy by the receive antenna 113 directly from the transmit antenna 111, thereby decoupling the antennas 111, 113 from one another.

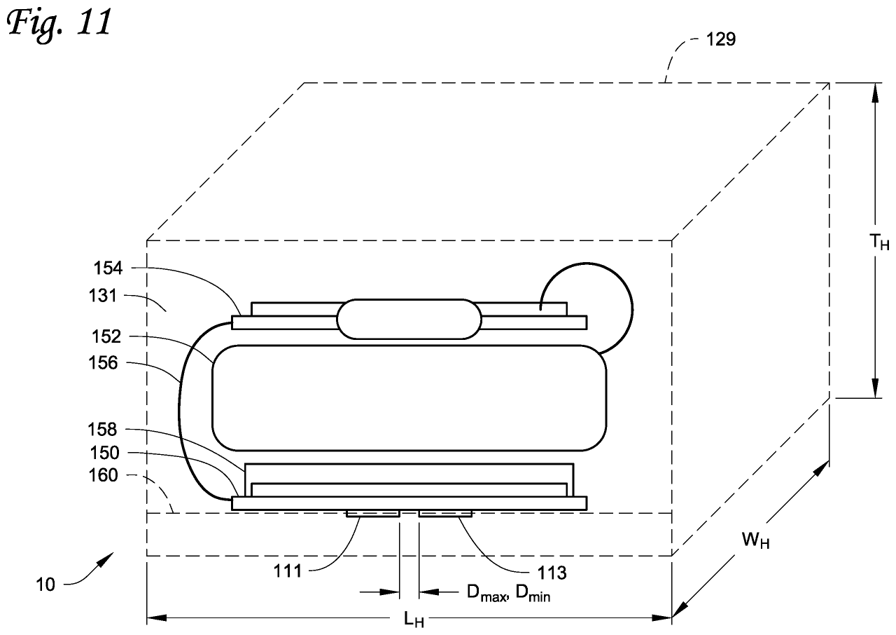

Referring to FIG. 11, an example configuration of the sensor 10 is illustrated. In FIG. 11, elements that are identical or similar to elements in FIG. 7 are referenced using the same reference numerals. In FIG. 11, the antennas 111, 113 are disposed on one surface of a substrate 150 which can be, for example, a printed circuit board. At least one battery 152, such as a rechargeable battery, is provided above the substrate 150, for providing power to the sensor 10. In addition, a digital printed circuit board 154 is provided on which the transmit circuit 115, the receive circuit 117, and the controller 119 and other electronics of the sensor 10 can be disposed. The substrate 150 and the digital printed circuit board 154 are electrically connected via any suitable electrical connection, such as a flexible connector 156. An RF shield 158 may optionally be positioned between the antennas 111, 113 and the battery 152, or between the antennas 111, 113 and the digital printed circuit board 154, to shield the circuitry and electrical components from RF interference.

As depicted in FIG. 11, all of the elements of the sensor 10, including the antennas 111, 113, the transmit circuit 115, the receive circuit 117, the controller 119, the battery 152 and the like are contained entirely within the interior space 131 of the housing 129. In an alternative embodiment, a portion of or the entirety of each antenna 111, 113 can project below a bottom wall 160 of the housing 129. In another embodiment, the bottom of each antenna 111, 113 can be level with the bottom wall 160, or they can be slightly recessed from the bottom wall 160.

The housing 129 of the sensor 10 can have any configuration and size that one finds suitable for employing in a non-invasive sensor device. In one embodiment, the housing 129 can have a maximum length dimension L_{H} no greater than 50 mm, a maximum width dimension W_{H} no greater than 50 mm, and a maximum thickness dimension T_{H} no greater than 25 mm, for a total interior volume of no greater than about 62.5 cm³.

In addition, with continued reference to FIG. 11 together with FIGS. 9A-9I, there is preferably a maximum spacing Dₘₐₓ and a minimum spacing Dₘᵢₙ between the transmit antenna 111 and the receive antenna 113. The maximum spacing Dₘₐₓ may be dictated by the maximum size of the housing 129. In one embodiment, the maximum spacing Dₘₐₓ can be about 50 mm. In one embodiment, the minimum spacing Dₘᵢₙ can be from about 1.0 mm to about 5.0 mm.

In operation, the sensor 10 is placed in relatively close proximity to the target. Relatively close proximity means that the sensor 10 can be close to but not in direct physical contact with the target, or alternatively the sensor 10 can be placed in direct, intimate physical contact with the target. The spacing between the sensor 10 and the target 107 can be dependent upon a number of factors, such as the power of the transmitted signal. Assuming the sensor 10 is properly positioned relative to the target 107, the transmit signal is generated, for example by the transmit circuit 115. The transmit signal is then provided to the transmit antenna 111 which transmits the transmit signal toward and into the target. A response resulting from the transmit signal contacting the analyte(s) is then detected by the receive antenna 113. The receive circuit 117 obtains the detected response from the receive antenna 113 and provides the detected response to the controller 119. The detected response can then be analyzed to detect at least one analyte. The analysis can be performed by the controller 119 and/or by the external device 125 and/or by the remote server 127.

The analysis can take a number of forms. In one embodiment the analysis can simply detect the presence of the analyte, i.e. is the analyte present in the target. Alternatively, the analysis can determine the amount of the analyte that is present.

The interaction between the transmitted signal and the analyte may, in some cases, increase the intensity of the signal(s) that is detected by the receive antenna, and may, in other cases, decrease the intensity of the signal(s) that is detected by the receive antenna. For example, in one non-limiting embodiment, when analyzing the detected response, compounds in the target, including the analyte of interest that is being detected, can absorb some of the transmit signal, with the absorption varying based on the frequency of the transmit signal. The response signal detected by the receive antenna may include drops in intensity at frequencies where compounds in the target, such as the analyte, absorb the transmit signal. The frequencies of absorption are particular to different analytes. The response signal(s) detected by the receive antenna can be analyzed at frequencies that are associated with the analyte of interest to detect the analyte based on drops in the signal intensity corresponding to absorption by the analyte based on whether such drops in signal intensity are observed at frequencies that correspond to the absorption by the analyte of interest. A similar technique can be employed with respect to increases in the intensity of the signal(s) caused by the analyte.

Detection of the presence of the analyte can be achieved, for example, by identifying a change in the signal intensity detected by the receive antenna at a known frequency associated with the analyte. The change may be a decrease in the signal intensity or an increase in the signal intensity depending upon how the transmit signal interacts with the analyte. The known frequency associated with the analyte can be established, for example, through testing of solutions known to contain the analyte. Determination of the amount of the analyte can be achieved, for example, by identifying a magnitude of the change in the signal at the known frequency, for example using a function where the input variable is the magnitude of the change in signal and the output variable is an amount of the analyte. The determination of the amount of the analyte can further be used to determine a concentration, for example based on a known mass or volume of the target. In an embodiment, presence of the analyte and determination of the amount of analyte may both be determined, for example by first identifying the change in the detected signal to detect the presence of the analyte, and then processing the detected signal(s) to identify the magnitude of the change to determine the amount.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A sensing method, comprising:
using a user wearable sensing assembly that includes a sensor removably mounted thereon to detect an analyte in a user when the user wearable sensing assembly is worn by the user, wherein the sensor includes at least one transmit antenna and at least one receive antenna, the at least one transmit antenna is positioned and arranged to transmit a signal into the user's body, wherein the signal is in a radio or microwave frequency range of the electromagnetic spectrum, and the at least one receive antenna is positioned and arranged to detect a response resulting from transmission of the signal by the at least one transmit antenna into the user's body;
installing the sensor at a mounting location of a non-user wearable sensing assembly that is configured to permit removable mounting of the sensor;
performing a detection using the sensor installed on the non-user wearable sensing assembly.

2. The sensing method of claim 1, comprising removing the sensor from a wrist strap of the user wearable sensing assembly.

3. The sensing method of claim 1, comprising using the non-user wearable sensing assembly to detect a characteristic of human tissue, animal tissue, plant tissue, an inanimate object, soil, a fluid, genetic material, or a microbe.

4. The sensing method of claim 1, wherein the analyte comprises cholesterol, glucose, alcohol, white blood cells, or luteinizing hormone.

5. The sensing method of claim 1, comprising detecting the analyte in interstitial fluid.

6. A sensing method, comprising:
using an in vivo sensing assembly that includes a sensor removably mounted thereon to detect an analyte in a user when the in vivo sensing assembly is worn by the user, wherein the sensor includes at least one transmit element and at least one receive element, the at least one transmit element is positioned and arranged to transmit a signal into the user's body, wherein the signal is in a radio or microwave frequency range of the electromagnetic spectrum, and the at least one receive element is positioned and arranged to detect a response resulting from transmission of the signal by the at least one transmit element into the user's body;
removably installing the sensor at a mounting location of an in vitro sensing assembly;
performing a detection using the sensor installed on the in vitro sensing assembly.

7. The sensing method of claim 6, comprising removing the sensor from a wrist strap of the in vivo sensing assembly.

8. The sensing method of claim 6, comprising using the in vitro sensing assembly to detect a characteristic of human tissue, animal tissue, plant tissue, an inanimate object, soil, a fluid, genetic material, or a microbe.

9. The sensing method of claim 6, wherein the analyte comprises cholesterol, glucose, alcohol, white blood cells, or luteinizing hormone.

10. The sensing method of claim 6, comprising detecting the analyte in interstitial fluid.
